(19) United States
(12) Patent Application Publication
(10) Pub. No.: US 2013/0012599 A1
(43) Pub. Date: Jan. 10, 2013

(54) SURFACE TREATMENT FOR GASIFIED CONSUMABLE MATERIALS

(76) Inventors: LYNN HESSON, Neenak, WI (US); Bruce Howe, Solana Beach, CA (US); Vincent Duprat, Encinitas, CA (US)

(21) Appl. No.: 13/197,709

(22) Filed: Aug. 3, 2011

Related U.S. Application Data
(60) Provisional application No. 61/505,057, filed on Jul. 6, 2011.

(51) Int. Cl. A61K 47/36; A23L 1/302; A23L 1/27; A23L 1/304; A23P 1/08; A23L 1/30; A23L 1/305

(52) U.S. Cl. 514/777; 426/89; 426/72; 426/99; 426/103; 426/93; 426/442; 426/302; 426/250

(57) ABSTRACT

A surface treatment for the preparation of gasified consumable materials comprising active ingredients. The surface treatment may comprise a combination of flavors, sweeteners, and active ingredients such as nutritional supplements and/or pharmaceuticals. The invention avoids the degradation of active ingredients during the manufacturing process since the active ingredients are applied to the surface of the composition and not cooked with the gasified consumable material. Methods for making and using the disclosed compositions are also within the scope of the invention.

This application claims priority from Provisional Application Ser. No. 61/505,057 filed Jul. 6, 2011, the entire contents of which are incorporated herein by reference.

### FIELD OF THE INVENTION

The invention generally relates to confectionery and formulations for dietary supplements, nutritional supplements, and pharmaceuticals.

### BACKGROUND OF THE INVENTION

Nutritional supplements provide an effective way of obtaining a balanced diet when an individual's normal meals do not meet healthy nutritional requirements. Nutritional supplements can be particularly important to children who often shun healthy foods such as fruits and vegetables. Nutritional supplements however often have an unappealing taste making it difficult to encourage children, and even adults, to use nutritional supplements with any regularity.

Gasified candy has been available for some time. For example, processes for making gasified candy are described in the following publications: U.S. Pat. Nos. 3,985,909, 3,985,910 and 4,001,457. U.S. Pat. No. 4,289,794 describes a method for preparing gasified candy whereby a sugar melt is gasified at superatmospheric pressure and then is cooled below its fusion temperature under superatmospheric pressure to form a gasified candy. As the gasified candy is wetted in the mouth the candy melts and the gas escapes producing an entertaining popping sensation. U.S. Pat. No. 4,263,328 describes a method of preparation of a tableted confection of gasified candy and mention that such candies may include a breath freshener. U.S. Pat. No. 4,271,206 describes gasified confection prepared by molding to a predetermined shape.

Gasified candies have also been formulated with nutritional and pharmaceutical ingredients. U.S. Patent Application No. US2004/0241209 to Kim discloses a gasified candy that is formulated with vitamins and amino acids. U.S. Pat. No. 7,887,832 to First et al. disclose a pharmaceutical composition comprising a pharmaceutical agent. The gasified compositions from these references are formulated by adding an active ingredient (i.e. vitamins, amino acids or a drug) to a hot melt of sugar and syrup which is then pressurized and cooled to produce crystals with a homogenous mixture of ingredients. This process however presents a significant problem in that cooking active ingredients in the sugar melt can destroy the activity of heat sensitive active ingredients. Vitamin C, for example, loses its antioxidant activity when exposed to heat (Food Chemistry Volume 128, Issue 1, 1 Sep. 2011, Pages 165-172).

What is needed in the art therefore is a technology for gasified nutrient and pharmaceutical compositions without exposing their active ingredients to heat degradation during the manufacturing process. What is also needed in the art is a surface treatment that provides gasified consumables (e.g. candy) with improved taste.

### SUMMARY OF THE INVENTION

Applicants discovered that gasified candy (and other consumable gasified materials), can achieve an improved, dynamic taste by providing the candy with a surface treatment. Applicants found that surface treatments can also be used to provide gasified materials with improved nutrient and/or pharmaceutical content by avoiding the cooking of active ingredients during the manufacturing process. The compositions of the invention find use in a variety of applications including nutritional, dental, pharmaceutical and cosmeceutical applications, for example.

A first objective of the invention is to provide a gasified nutrient composition comprising a gasified consumable material, and at least one bioactive agent on the surface of the gasified consumable material, wherein the interior of the gasified consumable material is free of bioactive agents.

A further objective of the invention is to provide a gasified nutrient composition comprising a gasified consumable material, and bioactive agents on the surface of the gasified consumable material, wherein the interior of the gasified consumable material is free of bioactive agents, and wherein the bioactive agents comprise a vitamin, mineral, antioxidant, protein, carbohydrate, amino acid, phytonutrient, fatty acid, or a combination thereof.

A further objective of the invention is to provide a gasified nutrient composition comprising a gasified consumable material, wherein the surface of the gasified consumable material comprises at least one bioactive agent, and wherein the interior of the gasified consumable material is essentially free of bioactive agents.

A further objective of the invention is to provide a gasified nutrient composition comprising a gasified consumable material, wherein the surface of the gasified consumable material comprises at least one bioactive agent, and wherein the interior of the gasified consumable material is essentially free of bioactive agents, and wherein the bioactive agents comprise a vitamin, mineral, antioxidant, protein, carbohydrate, amino acid, phytonutrient, fatty acid, or a combination thereof.

### DEFINITIONS

The term “gasified component,” or “consumable material” (i.e. GCM), refers to any edible, water soluble material that is capable of entrapping a pressurized gas. Consumable materials may comprise a crystalline material, such as a sugar (or sugars) for example.

The term “gasified” refers to the permeation of a material (e.g. consumable material) with a pressurized gas. Non limiting examples of gases suitable for the preparation of the inventive composition include, but are not limited to, carbon dioxide, nitrogen, air, helium, argon, neon, and combinations thereof. As used herein, the term “pressurized” means a pressure greater than 1 atmosphere.

The term “treating” refers to contacting a composition comprising at least one bioactive agent with a consumable (i.e. comestible) material such as a gasified consumable material (GCM), for example. The term “surface treatment,” or “surface treatment composition,” may also refer to the composition that is contacted with the gasified consumable material.

The term “nutrient” refers to any material that provides nourishment when consumed by a living organism. Examples of nutrients include, but are in no way limited to, vitamins, minerals, phytonutrients, amino acids, proteins, carbohydrates, plant extracts, fungal extracts, oils, fats, fatty acids, and combinations thereof.

The term “pharmaceutical,” or “drug,” refers to any chemical compound used in the diagnosis, treatment, or prevention of disease or other abnormal condition. Drugs include, but are not limited to, prescription and over-the-counter (OTC) drugs.

The term “biological effect” refers to the physiological effect that a nutrient or pharmaceutical imparts on a subject.

The term “effective amount,” or “therapeutically effective amount,” refers to an amount of an agent (e.g. bioactive agent) that is required to produce a desired biological effect in a target individual. One skilled in the art may readily determine such amounts based on the particular compound being administered, the bioavailability characteristics of the agent, the dose regimen, the age and weight of the individual, and other relevant factors. An effective amount may be, for example, a recommended daily allowance for a vitamin or mineral.

The term “wetting agent” refers to any material capable of allowing a powder and/or granulated material (e.g. surface treatment) to adhere to a GCM when applied according to the any of the processes disclosed herein. Wetting agents may be oil-, water- or emulsion-based. Wetting agents include, but are in no way limited to, palm oils, canola oils, coconut oils, vegetable oils, peanut oils, corn oils, soybean oils, coco butter, and combinations thereof. Wetting agents may comprise an bioactive ingredient, such as vitamins, minerals, a drug, or combination thereof.

### DETAILED DESCRIPTION

The invention generally relates to surface treatments for providing gasified consumable materials with improved taste and a nutrient and/or pharmaceutical effect.

In one aspect, the invention provides a gasified nutrient compositions comprising a gasified consumable material with at least one bioactive agent on the surface of the gasified consumable material, wherein the gasified consumable material is free of bioactive agents. The gasified consumable material (GCM) can be crystalline in structure and may be derived from any suitable crystal-forming material. In some aspects of the invention, crystalline GCMs are derived from, for example, one or more sugars, syrups, or combinations thereof. Suitable sugars for making the GCMs of the invention include glucose, fructose, sucrose, lactose, and combinations thereof (e.g. evaporated cane juice). It is also contemplated that these sugars may be combined with, for example, corn syrup, rice syrup (e.g. brown rice syrup), or a combination thereof. In a non-limited embodiment of the invention, GCMs comprise lactose and evaporated cane juice in crystallized form. Suitable sugar-based GCMs for use with the invention, and methods for their manufacture, are available in the art and include, but are not limited to, those disclosed in U.S. Pat. Nos. 3,985,909; 3,985,910; 4,001,457; 4,289,794; 4,263,328; 4,356,198; and 5,023,098, the entire contents of which are incorporated herein by reference.

Although specific sugars are called out above, one skilled in the art will appreciate that the invention may be practiced with any consumable material (e.g. crystalline material) that is capable of entrapping a pressurized gas (such as CO₂). For example, gasified consumable materials may be derived from a sugar substitute such as sorbitol, xylitol, and combinations thereof. Such sugar substitutes may be used alone, or in combination with one or more of the sugars and/or syrups disclosed herein.

The compositions of the invention may be produced by providing a GCM and contacting it with a surface treatment under conditions suitable to cause the surface treatment to adhere to the GCM. Surface treatments for practicing the invention may comprise at least one bioactive agent. As used herein, the term “bioactive agent” refers to any material that provides a nutrient, pharmaceutical and/or health benefit when consumed by an organism. Bioactive agents for use with a surface treatment include, but are not limited to, nutrients, pharmaceuticals, and combinations thereof.

Nutrients suitable for use in a surface treatment, include, but are not limited to, vitamins (e.g. vitamins B₆ and B folic acid, niacin, pantothenic acid, vitamins A, B1, B2, B3, B5, B6, B7, B9, B12, C, D, E and K, lipoic acid, biotin, co-enzymes B, M and Q, Glutathione, co-enzyme Q-10), minerals (e.g. iron, manganese, magnesium, copper, sulfur, zinc, calcium, selenium, iodine, sodium, potassium, chlorine, phosphorus, molybdenum), electrolytes, amino acids, proteins, melatonin, carotenoids (e.g. alpha- and beta-carotene, lutein, astaxanthin, lycopene, zeaxanthin), flavanoids (e.g. quercetin, anthocyanins, resveratrol, and the like), phenolic acids, citric acid, oxalic acid, phytie acid, essential fatty acids, plant extracts, fungal extracts, and combinations thereof.

Bioactive agents may be present in the surface treatment in a therapeutically effective amount. As one skilled in the art will appreciate, a therapeutically effective amount of a bioactive agent will depend upon the bioactive agent(s) that is selected and the amount of gasified nutrient composition that is to be consumed. The amount of bioactive agent(s) may be adjusted by varying the amount (i.e. thickness) of the surface treatment that is contacted with the gasified consumable material. The surface treatment may be contacted with the GCM in one or more applications.

The surface treatment may comprise any combination of materials that provides (for example) a desired flavor, scent, taste, texture and/or biological effect. Thus, the surface treatment may comprise bioactive agents, masking agents, coloring agents (e.g. dyes), binding agents, drying agents, gelatins, moisture barriers, plasticizers, surfactants, preservatives, or a combination thereof.

In aspects of the invention, the surface treatment (i.e. surface treatment composition) optionally comprises an effective amount of one or more masking agents for masking the taste of the bioactive agents (e.g. vitamins and minerals). Such masking agents include, but are not limited to, flavorings, sweeteners, debittering agents, and combinations thereof. One skilled in the art will appreciate that the ratio of masking agents to the bioactive agent(s) will vary depending upon the amount of the bioactive agent(s) that is present and the taste that is desired. It is also contemplated that gasified compositions (e.g. sugar-based crystals) may be given a desired flavor only by contacting the gasified composition with a surface treatment that is free of bioactive agents.

Suitable sweeteners for the surface treatment include, but are not limited to, sugars, artificial sweeteners, sugar substitutes, and combinations thereof. The amount of sweetener used in the composition will vary depending on the sweetness desired for the particular composition and the taste of the bioactive agent that is being masked.

Some sugar sweeteners for use with the invention include, but are not limited to, monosaccharides, disaccharides, polysaccharides and combinations thereof. Examples of suitable sugar sweeteners include but are not limited to isomalt, xylose, ribose, glucose, mannose, galactose, fructose, dextrose, sucrose, maltose, partially hydrolyzed starch or corn syrup solids and sugar alcohols such as sorbitol, xylitol, mannitol, glycerin and combinations thereof. Generally, and without being limited to any particular formulation, the total amount of sugar sweetener used may range between 0 and about 100 grams per 100 mL of surface treatment. In an aspect of the invention, the amount of sugar sweetener used may range between 0 grams and about 10 grams per 100 mL of surface treatment.

Artificial sweeteners may be used in place of, or in addition to, sugar sweeteners and/or sugar substitutes. Suitable artificial sweeteners for formulating surface treatments include, but are not limited to, aspartame, sucralose, cyclamates, saccharin, acesulfame K and mixtures thereof. As with other masking agents, the amount of artificial sweetener used in the composition will vary depending on the degree of sweetness desired for a particular composition. In one aspect of the invention, between about 0-100 grams of artificial sweetener is used per 100 mL of surface treatment. In another aspect of the invention, artificial sweetener is used in a range between about 0.45 to about 1.7 grams per 100 mL of surface treatment.

Sweeteners for use with the invention include sugar substitutes. As used herein, the term “sugar substitute” refers to any natural, non-toxic and non-sugar material capable of sweetening a surface treatment. Suitable sugar substitutes for use with the invention include, but are not limited to, stevia, mannitol, erythritol, lo han guo, and combinations thereof.

The surface treatment optionally comprises debittering agents, including, but not limited to, natural debittering agents, artificial debittering agents or debittering agents which inhibit a chemosensory response in the mouth or nose or mixtures thereof. Debittering agents for use in the present invention are commercially available, such as those marketed under the names Prosweet FL N&A K (by Virginia Dare), Bitterness Modifier 36734 (by Bush, Boake and Allen, Inc.), Natural Taste Masker 501.441/A and Special Taste Masker Compound 501.437/A (by Firmenich, Inc.), and may be identified by those skilled in the art.

The surface treatment may also comprise one or more flavoring agents. Suitable flavoring agents include natural flavors, artificial flavors, flavor enhancers and mixtures thereof. Natural and artificial flavors include, but are not limited to, mint (such as peppermint or spearmint), menthol, cinnamon, vanilla, artificial vanilla, fruit flavor, chocolate, artificial chocolate or bubblegum. Natural and artificial fruit flavors include, but are not limited to, cherry, grape, orange, strawberry or lemon. Flavor enhancers include, but are not limited to, citric acid. Flavoring agents are generally provided as a minor component of the surface treatment in amounts effective to provide a palatable flavor to the formulation. Flavoring agents may be present in the surface treatment in amounts that range between about 0.02 and 0.06 grams per 100 mL formulation. In one aspect of the invention, the surface treatment comprises about 25% flavoring agent.

The surface treatment may contain pH stabilizers, such as citric acid, added to stabilize the pH of the surface treatment and prevent microbial growth. Citric acid is advantageously added since a lower pH will prevent microbial growth and add to the stability of the final gasified nutrient composition. The surface treatment may similarly include preservatives. Preservatives useful in the present invention include, but are not limited to, sodium benzoate, potassium sorbate, salts of edetate (also known as salts of ethylenediaminetetraacetic acid, or EDTA, such as disodium edetate), parabens (such as methyl, ethyl, propyl and butyl p-hydroxybenzoic acids esters or mixtures thereof) or mixtures thereof. The amount of preservative needed will be readily determined according to knowledge that is generally available in the art.

Coloring agents also may be incorporated into the surface treatment. Such coloring agents should be selected to avoid chemical incompatibilities the other ingredients (e.g. bioactive agents) in the formulation. Suitable coloring agents for use with the compositions are well known to those skilled in the art and include, for example, artificial and natural coloring agents such as vegetable dyes, for example.

Gasified consumable materials may be contacted with a surface treatment using any method that allows the surface treatment to adhere to the surface of the gasified consumable material. Suitable contacting methods include, but are not limited to, spray coating, wet granulation, tumbling, pan coating (e.g. hot or cold pan coating), brush coating, padding, fogging and combinations thereof. The order in which the various components of the surface treatment are applied, may vary. For example, the surface treatment may be applied as a single (complete) composition, or the components of the surface treatment may be applied individually in series. The surface treatment (and/or surface treatment components) may be applied one or a number of times depending on the amount of surface treatment desired. The surface treatment may be applied as a liquid, powder, grain, foam, mist or combination thereof.

While the invention is disclosed as having a surface treatment comprising at least one bioactive agent, it is also contemplated that the surface treatment may be free of bioactive agents. Such surface treatments may be desirable for simply adjusting the flavor of the gasified consumable material. For example, the surface treatment may be formulated with sweeteners and flavorings to provide a gasified confection that comprises one flavor provided by the GCM, and an additional flavor(s) provided by the surface treatment.

One aspect of the invention concerns the amount of surface treatment that is contacted with the gasified consumable material. The amount of surface treatment used may range between an amount that is insufficient to coat the entire surface of the gasified consumable material (i.e. dusted with surface treatment) and amount that is sufficient to coat the entire gasified consumable material such that a crust is formed around the gasified consumable material. Of course, the amount of surface treatment will depend on the dosage and concentration that is desired for the bioactive agent. In some embodiments, the gasified nutrient composition has a proportionately larger amount of GCM (by weight) than surface treatment. Suitable weight ratios for the surface treatment relative to gasified consumable materials (i.e. surface treatment/gasified consumable material) include, but are not limited to, about the following ranges, 50/50, 40/60, 30/70, 20/80, 10/90, 5/95 and less than about 5% surface treatment. In a non-limiting embodiment of the invention, between about 0-100 grams of evaporated cane juice (i.e. GCM) is used per 100 mL of surface treatment.

In some aspects of the invention, the presence of bioactive agents is limited to the surface of the gasified consumable material. That is, the GCM may be free of bioactive agents. This is accomplished by omitting bioactive agents from the formulation and manufacturing of the GCM prior to contacting the GCM with the surface treatment. This particular formulation provides improved bioavailability and taste as the bioactive agent is first introduced to the oral mucosa before the GCM (e.g. sugar) can adjust the osmolality of the mouth. Omitting bioactive agents from the GCM also permits the GCM to be formulated without any bitterness so that sugars in the GCM further mask any poor taste by the initial dissolving of the bioactive agent which coats the GCM. Omitting bioactive agents from the GCM also allows the GCM (e.g. sugar melt) to be cooked without exposing the bioactive agents to high heat and degradation.

In an aspect of the invention, a gasified nutrient composition is produced by contacting a gasified consumable material with a surface treatment. For example, gasified sugar crystals may be obtained according to any of the processes disclosed in U.S. Pat. No. 3,985,909, 3,985,910, 4,001,457, 4,289,794, 4,263,328, 4,356,198, or 5,023,098. Such crystals may then be fractured and screened to obtain crystals of a desired size. The crystals are then contacted with a surface treatment of the present invention. Such contacting may be accomplished, for example, by placing the gasified crystal particles in a pan-type, open-faced mixer and spraying them with a suitable wetting agent, such as sunflower oil for example. Once coated with oil, the crystals are contacted with a surface treatment comprising bioactive agents, such as vitamins and minerals, and optionally one or more of a flavor, sweetener, debittering agent, or combination thereof (the bioactive agent may assume any suitable texture that permits the bioactive agent to adhere to the wetting agent and crystals; the bioactive agent may be powdered, granulated, or combination thereof, for example). The crystals are then permitted to cool and dry before they are packaged. The crystals are then ready to be consumed as a dietary and nutritional supplement that provides an entertaining popping sensation when eaten. The crystals may also be used as a food additive to supplement food with nutritional and/or pharmaceutical agents.

One aspect of the invention relates to a method of making a gasified nutrient composition. The invention can be practiced in such aspects by providing a gasified consumable material, such as crystals for example. The gasified consumable material, or crystals, are then contacted with a composition (i.e. surface treatment) comprising at least one bioactive agent, such as a vitamin and/or mineral formulation. The surface treatment may be contacted with the GCM by the use of a wetting agent. A wetting agent, such as palm oil, is contacted with the GCM to provide a coating. The wetted GCM is then contacted with a surface treatment that may be in the form of grains, a powder, or a combination thereof. The wetting agent thus causes the surface treatment to adhere to the GCM in a desired amount.

It is within the scope of the invention to provide bioactive agents within the wetting agent. For example, the wetting agent may comprise palm oil and a combination of vitamins and/or minerals. Such wetting agents may be used in combination with surface treatment compositions (e.g. powders and grains) that are either free of, or supplemented with, one or more bioactive agents. It should be noted that bioactive agents may be micro encapsulated so as to mask the taste of the bioactive agents. Technology for such microencapsulation is readily available in the art and includes, by way of example only, the processes and materials disclosed in the following publications, the disclosures of which are incorporated by reference in their entirety: U.S. Pat. App. No. 2005/0136114, and U.S. Pat. Nos. 6,139,865 and 6,451,345.

In order to further illustrate the present invention and the advantages thereof, the following specific example is given, it being understood that this example is intended to only be an illustration without serving as a limitation on the scope of the claimed invention.

### Example

A gasified consumable nutrient composition was produced by mixing evaporated cane juice, lactose, rice syrup solids, and water. The mixture was cooked to temperature and color was added. The mixture was pressurized with carbon dioxide and allowed to cool. Cooling of the mixture resulted in crystallization of the mixture which was then collected and screened to produce crystals of appropriate size. The crystals were then placed in a tumbler in a controlled environment (below 65F and 35% RH). The crystals were combined with melted palm oil and tumbled with a mixture of vitamins, minerals, flavors, and color. The crystals were tumbled until the mixture had set on the crystals.

We claim:

1. A gasified nutrient composition comprising:
a) a gasified consumable material; and
b) a surface treatment in contact with the surface of the gasified consumable material, wherein the surface treatment comprises at least one bioactive agent.

2. The gasified nutrient composition of claim 1, wherein the at least one bioactive agent comprises a vitamin, mineral, protein, amino acid, plant extract, fatty acid, oil, pharmaceutical, or combination thereof.

3. The gasified nutrient composition of claim 2, wherein the at least one bioactive agent comprises at least one vitamin and/or at least one mineral.

4. The gasified nutrient composition of claim 1, wherein the interior of the gasified consumable material is free of any vitamin, mineral, protein, amino acid, plant extract, fatty acid, oil, pharmaceutical or combination thereof.

5. The gasified nutrient composition of claim 1, further a wetting agent at the interface between the gasified consumable material and the surface treatment.

6. The gasified nutrient composition of claim 5, wherein the wetting agent comprises at least one bioactive agent.

7. The gasified nutrient composition of claim 1, wherein the gasified consumable material comprises gasified crystals.

8. The gasified nutrient composition of claim 7, wherein said gasified crystals comprise sugar, evaporated cane juice, syrup, or combination thereof.

9. The gasified nutrient composition of claim 1, wherein said gasified nutrient composition is in contact with grains, syrup, paste, powder, or combination thereof.

10. The gasified nutrient composition of claim 1, wherein the surface treatment comprises at least one of a flavoring, fragrance, color and sweetener.

11. A gasified nutrient composition made according to the process comprising:
a) providing a gasified consumable material; and
b) contacting the gasified consumable material with a surface treatment, wherein the surface treatment comprises at least one bioactive agent.

12. The gasified nutrient composition of claim 11, wherein the at least one bioactive agent comprises a vitamin, mineral, protein, amino acid, plant extract, fatty acid, oil, pharmaceutical, or combination thereof.

13. The gasified nutrient composition of claim 12, wherein the at least one bioactive agent comprises at least one vitamin and/or at least one mineral.

14. The gasified nutrient composition of claim 12, wherein the interior of the gasified consumable material is free of any vitamin, mineral, protein, amino acid, plant extract, fatty acid, oil, pharmaceutical, or combination thereof.

15. The gasified nutrient composition of claim 11, wherein the surface treatment is contacted with the surface of the gasified consumable material after the gasified consumable material is contacted with a wetting agent.

16. The gasified nutrient composition of claim 15, wherein the wetting agent comprises at least one bioactive agent.

17. The gasified nutrient composition of claim 11, wherein the gasified consumable material comprises gasified crystals.

18. The gasified nutrient composition of claim 16, wherein said gasified crystals comprise sugar, evaporated cane juice, syrup, or combination thereof.

19. The gasified nutrient composition of claim 11, wherein said gasified nutrient composition is in contact with a syrup, paste, powder, grains, or combination thereof.

20. The gasified nutrient composition of claim 11, wherein the surface treatment comprises at least one of a flavoring, fragrance, color and sweetener.
